(19) United States
(12) Patent Application Publication
(10) Pub. No.: US 2008/0110763 A1
(43) Pub. Date: May 15, 2008

(54) Method For Producing Alkoxylated 2,5-Dihydrofuran But-2-Ene Derivatives Or Tetra-1,1,4,4-Alkoxylated But-2-Ene Derivatives

(75) Inventors: Ingo Richter, Schwetzingen (DE); Hermann Putter, Neustadt (DE); Ulrich Griesbach, Mannheim (DE); Nils Bottke, Mannheim (DE)

Correspondence Address: OBLON, SPIVAK, MCCLELLAND MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/908,506

(22) PCT Filed: Mar. 23, 2006
(86) PCT No.: PCT/EP06/60989

§ 371 Date: Sep. 13, 2007

(30) Foreign Application Priority Data
Mar. 24, 2005 (DE) 10 2005 013 631.1

(51) Int. Cl. C25D 3/62; C07D 307/02

(52) U.S. Cl. 205/247; 549/476

(57) ABSTRACT

A process for the preparation of 2,5-dihydrofuran derivatives substituted in the 3- or 4-position, which in the 2- or in the 5-position or at both positions each carry a C₁- to C₆-alkoxy radical (DHF-alkoxy derivatives 1), or 1,1,4,4-tetraalkoxy-but-2-ene derivatives substituted in the 3- or 4-position, from 2-butene-1 ,4-diol derivatives of the general formula (I)

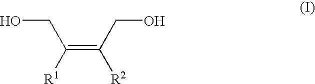

in which the radicals R¹ and R² independently of one another are hydrogen, C₁- to C₆-alkyl, C₆- to C¹²-aryl or C₅- to C₁₂-cycloalkylene or

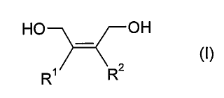

R¹ and R², together with the double bond to which they are bonded, form a C₆- to C₁₂-aryl radical or a mono- or polyunsaturated C₅- to C₁₂-cycloalkyl radical,

or

from their mixture with 2,5-dihydrofuran derivatives substituted in the 3- position or 4-position, which in the 2- or in the 5-position carry a C₁- to C₆-alkoxy radical, by electro-chemical oxidation in the presence of a C₁- to C₆-monoalkyl alcohol.

Process for the preparation of alkoxylated 2,5-dihydrofuran or tetra-1,1,4,4-alkoxylated but-2-ene derivatives

Description

The invention relates to a novel process for the preparation of 2,5-dihydrofuran derivatives substituted in the 3- or 4-position, which in the 2- or in the 5-position or at both positions each carry a C₁-C₆-alkoxy radical, or 1,1,4,4-tetraalkoxy-but-2-enes substituted in the 3- or 4-position (DHF-alkoxy derivatives).

In the case of the dihydrofurans, the naming of the atom positions in the ring takes place according to the customary nomenclature rules as in formula (V).

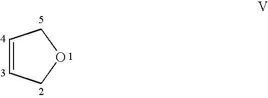

In the case of the fused dihydrofurans, the naming of the atom positions of the atoms belonging to the furan ring changes according to the customary nomenclature rules, as is intended to be shown by the example of the isobenzofuran as in formula (VI)

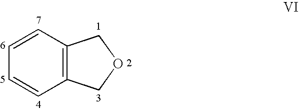

In this text, for reasons of better clarity, contrary to the abovementioned rule for the fused ring systems and in particular of isobenzofuran, m the naming of the atom positions as is customary in nonfused furan rings is also retained in compounds in which the furan ring is present in fused form. In this text, the naming of the atom positions in benzo-fused dihydrofuran ring systems thus takes place as in formula (VII).

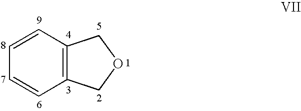

The electrochemical synthesis of 2,5-dihydro-2,5-dimethoxyfuran starting from furans is already known.

Thus, DE-A-27 10 420 and DE-A-848 501 describe the anodic oxidation of furans in the presence of sodium bromide or ammonium bromide as conductive salts.

Furthermore, the cyanide-catalysed anodic oxidation of furans is known from Bull. Chem.Soc. Jpn. 60, 229-240, 1987. EP-A-078 004 discloses the anodic oxidation of furans using alcolates, halides and sulfonates as conductive salts, while WO 2004/85710 describes the direct anodic oxidation of furans on special boron-doped diamond electrodes.

The alkoxylation of unsubstituted 2,5-dihydrofuran by electrochemical oxidation is disclosed in EP-A-78004. Substituted furans are electrochemically oxidized in DE 103 24 192. Higher raw material prices and increased expenditure on cooling caused by the boiling point of the dihydrofuran derivatives lead to unsatisfactory economy of the processes.

It was therefore the object to make available an electrochemical process for the preparation of alkoxylated 2,5-dihydrofuran or tetra-1,1,4,4-alkoxybut-2-ene derivatives, which is economical and makes the desired products available in high yields and with good selectivity.

Accordingly, a process has now been found for the preparation of 2,5-dihydrofuran derivatives substituted in the 3- or 4-position, which in the 2- or in the 5-position or at both positions each carry a C₁- to C₆-alkoxy radical, or 1,1,4,4-tetraalkoxy-but-2-ene derivatives substituted in the 3- or 4-position (DHF-alkoxy derivatives), from 2-butene-1,4-diol derivatives of the general formula (I)

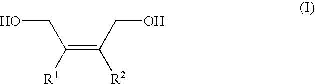

in which the radicals R¹ and R² independently of one another are hydrogen, C₁- to C₆-alkyl, C₆- to C₁₂-aryl such as, for example, phenyl or C₅- to C₁₂-cycloalkyl, or R¹ and R², together with the double bond to which they are bonded, form a C₆- to C₁₂-aryl radical such as, for example, phenyl, mono- or poly-C₁- to C₆-alkyl, halogen- or alkoxy-substituted phenyl, or a mono- or polyunsaturated C₅- to C₁₂-cycloalkyl radical, or

a mixture of the 2-butene-1,4-diol derivatives of the formula (I) with 2,5-dihydrofuran derivatives substituted in the 3- or 4-position of the formula (II), which in the 2- or in the 5-position carry a C₁- to C₆- alkoxy radical, by electrochemical oxidation in the presence of a C₁- to C₆-monoalkyl alcohol.

The C₁- to C₆-monoalkyl alcohol preferably employed is methanol or isopropanol.

Particularly preferably, the process according to the invention is employed for the preparation of

1. DHF-alkoxy derivatives of the general formula (II),

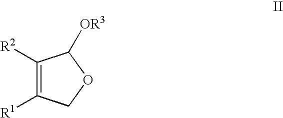

in which the radicals R¹, R² and R³ have the following meaning: R¹, R² independently of one another are hydrogen, C₁- to C₆-alkyl, C₆- to C₁₂-aryl or C₅- to C₁₂-cycloalkyl,
or
R¹ and R², together with the double bond to which they are bonded, form a C₆- to C₁₂-aryl radical or a mono- or polyunsaturated C₅- to C₁₂-cycloalkyl radical, R³ is C₁- to C₆-alkyl, prepared from 2-butene-diol derivatives of the formula (I) by electrochemical oxidation in the presence of a C₁- to C₆-monoalkyl alcohol.

2. DHF-alkoxy derivatives of the general formula (III),

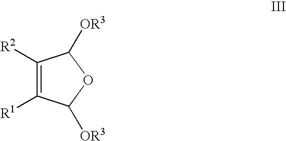

in which the radicals R¹, R² and R³ have the same meaning as in the general formula (II)
from 2-butenediol derivatives of the formula (I) or their mixture with DHF-alkoxy derivatives of the general formula (II)
or

3. 1,1,4,4,-Tetraalkoxy-but-2-ene derivatives substituted in the 3- or 4-position of the general formula (IV),

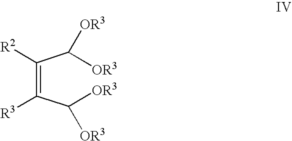

in which the radicals R¹, R² and R³ have the same meaning as indicated above in the general formula (II), from 2-butene-diol derivatives of the formula (I).

The process according to the invention is particularly suitable for the preparation of

1a. DHF-alkoxy derivatives of the general formula (IIIa)

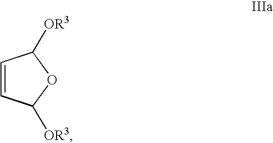

in which R³ is C₁- to C₆-alkyl, from butene-1,4-diol of the general formula (I), where R¹ and R² in formula (I) are hydrogen.

In comparison to the furan used as a starting material in the processes of the prior art, 2-butene-1,4-diol is significantly less expensive. On account of a higher boiling point of 2-butene-1,4-diol, the expenditure on cooling during the reaction is moreover reduced and higher reaction temperatures are possible. A significant further advantage of this starting material is its markedly lower toxicity. Preferably, cis-butene-1,4-diol or diastereomer mixtures comprising at least 20% by weight of cis-butene-1,4-diol are employed in the process according to the invention.

2a. The process according to the invention is particularly suitable for the preparation of DHF-alkoxy derivatives of the general formula (IIIb),

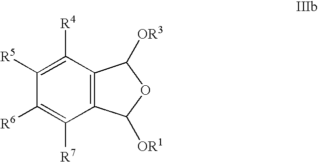

in which the radicals R⁴, R⁵, R⁶ and R⁷ are hydrogen, C₁- to C₄-alkyl, C₁- to C₆-alkoxy or halogen, and R³ has the meaning indicated in the general formula (II),
from the 2-butene-1,4-diol derivatives substituted in the 3 or 4-position, of the general formula (Ia),

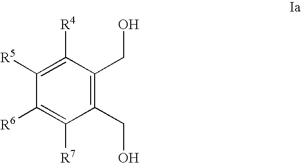

in which the radicals R⁴, R⁵, R⁶ and R⁷ are hydrogen, C₁- to C₄-alkyl, C₁- to C₆-alkoxy or halogen,
or
from the mixture of the 2-butene-1,4-diol derivatives substituted in the 3 or 4-position, of the general formula (Ia) and the DHF-alkoxy derivatives of the general formula (II),
or

3a. 1,1,4,4,-Tetraalkoxy-but-2-ene derivatives of the general formula (IVa),

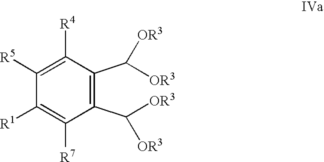

in which the radicals R⁴, R⁵, R⁶ and R⁷ are hydrogen, C₁- to C₄-alkyl, C₁- to C₆-alkoxy or halogen, and R³ has the meaning indicated in the general formula (II),
from the in butene-1,4-diol derivatives of the general formula (Ia) or their mixture with the DHF-alkoxy derivatives of the general formula (II).

Very particularly preferably, in the compounds of the general formulae (Ia), (IIIb) and (IVa) the radicals R⁴, R⁵, R⁶ and R⁷ are hydrogen.

In general, the compounds of the general formulae (II), (III) and (IV) are obtained in the form of their mixtures. These mixtures can be worked up with the aid of generally known separation methods.

It is also preferred, if the desired target products are a compound of the general formula (III) or (IV), to start from 2-butene-1,4-diol derivatives of the general formula (I). From the reaction mixture resulting here, the compound of the general formula (II) not desired is fed back into the electrolysis cell and then serves, together with the corresponding 2-butene-1,4-diol derivative of the general formula (I), as a primary product for the preparation of the target products having the desired higher number of alkoxy radicals.

In the electrolyte, the C₁- to C₆-mono alcohol, based on the 2-butene-1,4-diol derivative of the general formula (i), is employed in an equimolar amount or in an excess of up to 1:20 and then simultaneously serves as a solvent or diluent for the compound of the general formula (II) and the compound of the general formula (I) formed. Preferably, a C₁- to C₆-monoalkyl alcohol and very particularly preferably methanol is employed.

If appropriate, customary cosolvents are added to the electrolysis solution. These are the inert solvents having a high oxidation potential generally customary in organic chemistry. By way of example, dimethylformamide, dimethyl carbonate or propylene carbonate may be mentioned.

Conductive salts which are comprised in the electrolysis solution are in general at least one compound selected from the group potassium, sodium, lithium, iron, alkali metal, alkaline earth metal and tetra(C₁- to C₆-alkyl)ammonium, preferably tri(C₁- to C₆-alkyl)methylammonium, salts. Suitable counterions are sulfate, hydrogensulfate, alkyl-sulfates, arylsulfates, halides, phosphates, carbonates, alkylphosphates, alkylcarbonates, nitrate, alcoholates, tetrafluoroborate or perchlorate.

Furthermore, suitable conductive salts are the acids derived from the abovementioned anions.

Methyltributylammonium methylsulfate (MTBS), methyltriethylammonium methylsulfate or methyltripropylmethylammonium methylsulfate are preferred.

In addition, suitable conductive salts are also ionic liquids. Suitable ionic liquids are described in “Ionic Liquids in Synthesis”, eds. Peter Wasserscheid, Tom Welton, Verlag Wiley VCH, 2003, Chap. 3.6, pages 103-126.

The pH of the electrolyte is adjusted to a pH in the range from 2 to 7, preferably 2.5 to 5, by addition of organic and inorganic acids such as, for example, citric acid, tartaric acid, sulfuric acid, phosphoric acid, sulfonic acids, C₁- to C₆-carboxylic acids such as formic acid, acetic acid, propionic acid or by use of buffer systems known per se.

The process according to the invention can be carried out in all customary types of electrolysis cells. Preferably, it is carried out continuously using undivided flow cells. Very particularly suitable are bipolar-switched capillary gap cells or stacked plate cells, in which the electrodes are designed as plates and are arranged plane-parallel (cf. Ullmann's Encyclopedia of Industrial Chemistry, 1999 electronic release, Sixth Edition, VCH-Verlag Weinheim, Volume Electrochemistry, Chapter 3.5. special cell designs, and Chapter 5, Organic Electrochemistry, Subchapter 5.4.3.2 Cell Design). Such electrolysis cells are, for example, also described in DE-A-19533773.

The current densities at which the process is carried out are in general 1 to 20, preferably 3 to 5, mA/cm². The temperatures are customarily −20 to 55° C., preferably 20 to 40° C. In general, the process is carried out at normal pressure. Higher pressures are preferably used, if it is intended to work at relatively high temperatures, in order to avoid boiling of the starting compounds or cosolvents.

Suitable anode materials are, for example, noble metals such as platinum or metal oxides such as ruthenium or chromium oxide or mixed oxides of the type RuoₓTiOₓ. Graphite or carbon electrodes are preferred. Anodes having diamond surfaces are furthermore preferred.

At the cathode, different electrochemical reductions can be carried out on organic compounds. Such reductions are described, in particular, in DE-A-10058304. In general, however, hydrogen is evolved at the cathode by electrochemical reduction of protons or alcohol.

Suitable cathode materials are, for example, iron, steel, stainless steel, nickel or noble metals such as platinum and also graphite or carbon materials, graphite being preferred. Cathodes having diamond surfaces are furthermore preferred.

The system graphite as anode and cathode, and graphite as anode and nickel, stainless steel or steel as cathode, is particularly preferred. Anodes having diamond surfaces are furthermore preferred.

After completion of the reaction, the electrolysis solution is worked up according to general separation methods. For this, the electrolysis solution is in general first brought to a pH from 8 to 9, then distilled and the individual compounds are obtained separately in the form of different fractions. A further purification can be carried out, for example, by crystallization, distillation or by chromatography. If 2,5-dimethoxytetrahydrofuran is to be prepared from 2,5-dihydro-2,5-dimethoxyfuran, a purification is not necessary and the crude product obtained by the process according to the invention can be employed.

Experimental Section

### EXAMPLE 1

### 2,5-dimethoxy-2,5-dihydrofuran

| | |
|---|---|
| Apparatus: | Undivided stacked plate cell having 6 graphite |
| | electrodes (65 mm Ø, gap: 1 mm) |
| Anode and | Graphite |
| cathode: | |
| Electrolyte: | 72.6 g of 2-butene-1,4-diol |
| | 25.7 g of methyltributylammonium methylsulfate |
| | (MTBS) 1.4 g of H₃PO₄, 96% strength |
| | 660.0 g of methanol |
| Cathode: | Graphite |

Electrolysis using 4.8 F./mol of 2-butene-1,4-diol

| | |
|---|---|
| Current density: | 3.4 A dm⁻² |
| Temperature: | 22° C. |

During the electrolysis under the conditions indicated, the electrolyte was pumped through the cell via a heat exchanger at a flow rate of 200 I/h for 19h.

After completion of the electrolysis, the discharge from the electrolysis was adjusted to pH 8 to 9 by addition of 1.89 g of sodium methoxide (30% strength in methanol), freed from the methanol by distillation and the residue was distilled at 70° C. and 1 mbar. In 35 this process, 47.9 g, corresponding to a yield of 46%, of 2,5-dimethoxy-2,5-dihydro-furan was obtained. The selectivity was 51%.

### EXAMPLE 2

### 1,3-dimethoxy-1,3-dihydroisobenzofuran

| | |
|---|---|
| Apparatus: | Undivided stacked plate cell having 6 graphite |
| | electrodes (65 mm Ø, gap: 1 mm) |
| Anode: | Graphite |
| Electrolyte: | 35.0 g of 1,2-benzenedimethanol |
| | 2.3 g of MTBS (60% strength in methanol) |
| | 2.2 g of H₂SO₄, 96% strength |
| | 660.5 g of methanol |
| Cathode: | Stainless steel foil on graphite |

Electrolysis using 9.5 F./mol of 1,2-benzenedimethanol

| | |
|---|---|
| Current density: | 3.4 A dm⁻² |
| Temperature: | 20° C. |

During the electrolysis under the conditions indicated, the electrolyte was pumped through the cell via a heat exchanger at a flow rate of 200 I/h for 12 h.

After completion of the electrolysis, the discharge from the electrolysis was adjusted to pH 8 to 9 by addition of 4.3 g of sodium methoxide (30% strength in methanol), freed from the MeOH by distillation, treated with 150 ml of methyl tert-butyl ether, the precipitated conductive salt was filtered off with suction through a pressure suction filter and the filtrate was distilled at 70° C. and 1 mbar. In this process, 3.4 g (corresponding to a 9% yield ) of 1-methoxy-1,3-dihydroisobenzofuran, 14.4 g (corresponding to a 31.7% yield) of 1,3-dimethoxy-1,3-dihydroisobenzofuran and 4.1 g (corresponding to a 20.4% yield ) of o-phthalaldehyde tetramethyl acetal were obtained. The 1-methoxy-1,3-dihydroisobenzofuran could be used again for an electrolysis.

1. A process for the preparation of 2,5-dihydrofuran derivatives substituted in the 3- or 4-position, which in the 2- or in the 5-position or at both positions each carry a C₁- to C₆-alkoxy radical (DHF-alkoxy derivatives), or 1,1,4,4-tetraalkoxy-but-2-ene derivatives substituted in the 3- or 4-position, from 2-butene-1,4-diol derivatives of the general formula (I) 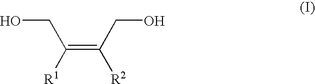
in which the radicals R¹ and R² independently of one another are hydrogen, C₁- to C₆-alkyl, C₆- to C₁₂-aryl or C₅- to C₁₂-cycloalkylene or R¹ and R², together with the double bond to which they are bonded, form a C₆- to C₁₂-aryl radical or a mono- or polyunsaturated C₅- to C₁₂-cycloalkyl radical,
or
from their mixture with 2,5-dihydrofuran derivatives substituted in the 3- or 4-position, which in the 2- or in the 5-position carry a C₁- to C₆-alkoxy radical, by electrochemical oxidation in the presence of a C₁- to C₆-monoalkyl alcohol.

2. The process according to claim 1, where DHF-alkoxy derivatives of the general formula (II) 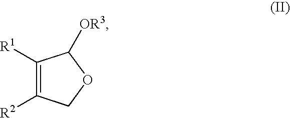
in which R¹and R² independently of one another are hydrogen, C₁- to C₆-alkyl, C₆- to C₁₂-aryl or C₅- to C₁₂-cycloalkyl,
or
R¹ and RW, together with the double bond to which they are bonded, form a C₆- to C₁₂-aryl radical or a mono- or polyunsaturated C₅- to C₁₂-cycloalkyl radical, R³ is C₁- to C₆-alkyl, are prepared from 2-butene-1,4-diol derivatives of the formula (I) by electrochemical oxidation in the presence of a C₁- to C₆-monoalkyl alcohol.

3. The process according to claim 2, where DHF-alkoxy derivatives of the general formula (III) 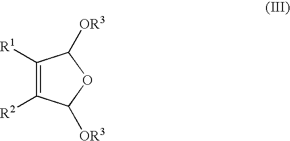
in which the radicals R¹, R² and R³ have the meaning indicated in formula (II), are prepared from 2-butene-1,4-diol derivatives of the formula (I) or their mixture with DHF-alkoxy derivatives of the general formula (II).

4. The process according to claim 3, where 1,1,4,4-tetraalkoxy-but-2-ene derivatives substituted in the 2- or 4-position of the general formula (IV) 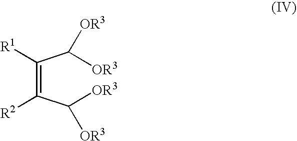
in which the radicals R¹, R² and R³ have the meaning mentioned above in formula (II), are prepared from 2-butene-1,4-diol derivatives of the general formula (I) or their mixture with DHF-alkoxy derivatives of the general formula (III).

5. The process according to claim 1, where the aliphatic C₁- to C₆-monoalkyl alcohol is methanol or isopropanol.

6. The process according to claim 1, wherein, per mol of butene-1,4-diol derivative of the general formula (I), at least 1 mol of monoalkyl alcohol is employed.

7. The process according to claim 1, where the process is carried out in an electrolyte which, as a conductive salt, comprises sodium, potassium, lithium, iron and tetra (C₁- to C₆-alkyl)ammonium salts with sulfate, hydrogensulfate, alkylsulfates, arylsulfates, halides, phosphates, carbonates, alkylphosphates, alkylcarbonates, nitrate, alcoholates, tetrafluoroborate, hexafluorophosphate or perchlorate as a counterion or ionic liquids.

8. The process according to claim 7, wherein the electrolyte used comprises less than 20% by weight of water.

9. The process according to claim 7, wherein the pH of the electrolyte is kept in a range from 2.5 to 5 by addition of sulfuric acid, phosphoric acid, sulfonic acid, C₁- to C₆-carboxylic acid or by use of a buffer system.

10. The process according to claim 1, which is carried out in a bipolar-switched capillary gap cell or stacked plate cell or in a divided electrolysis cell.
